(19) United States
(12) Patent Application Publication
(10) Pub. No.: US 2019/0221319 A1
(43) Pub. Date: Jul. 18, 2019

(54) SYSTEM AND METHOD FOR PROVIDING WORKFLOW-DRIVEN COMMUNICATIONS IN AN INTEGRATED SYSTEM

(71) Applicant: Spok, Inc., Springfield, VA (US)

(72) Inventors: Luke Bonde, Eden Prairie, MN (US); Alex Conway, Shakopee, MN (US); Scott Diestler, Cottage Grove, MN (US); Claire Farney, Hopkins, MN (US); Joe Hess, Farmington, MN (US); Michael Jenkins, St Johns, FL (US); Adam Lund, Saint Louis Park, MN (US); David Meyer, Bloomington, MN (US); Adam Nunez Frausto, Minneapolis, MN (US); Jeff Rapp, Minneapolis, MN (US); John Rolf, Plymouth, MN (US); Michael Schmid, Jordan, MN (US); Bobby Schmitt, Eden Prairie, MN (US); Austin Schuch, Eden Prairie, MN (US); Mclanahan Stevens, Chaska, MN (US); Michelle Thorsell, Plymouth, MN (US)

(21) Appl. No.: 16/251,532

(22) Filed: Jan. 18, 2019

Related U.S. Application Data
(60) Provisional application No. 62/619,012, filed on Jan. 18, 2018.

(51) Int. Cl. G16H 80/00; G16H 40/20; G06Q 10/10

(52) U.S. Cl. CPC G16H 80/00; G01S 19/01; G06Q 10/103; G16H 40/20

(57) ABSTRACT

According to one embodiment, a method of initiating and/or routing communications within a system comprised of a plurality of disparate sub-systems is provided. The method includes collecting information from one or more sub-systems regarding participants in the integrated system, wherein collected information includes one or more of schedule, status, and contact information, wherein the collected information is stored in a central directory. A communication initiated by one of the sub-systems is received, and an event is identified within the received communication. A workflow plan is initiated based on the identified event, wherein the workflow plan utilizes collected information to generate workflow-driven communications to one or more participants. The workflow-driven communications is then delivered to the one or more participants.

### TECHNICAL FIELD

This invention relates generally to integrated communications systems, and in particular to workflow-driven communications in integrated communications systems.

### BACKGROUND

In hospital applications, the timeliness with which data is analyzed and communicated to participant (e.g., doctors, nurses, patients) can be critical in patient outcomes. A typical hospital relies on communication infrastructure (e.g., paging systems, email, text message systems) to implement this communication, but relies on the hospital staff to implement how communications are initiated and to who. For example, a patient requesting assistance in a room may hit a call button that generates an alert at the nurse's station. However, the call is a simple one-way communication, the response to which is handled by hospital protocol. For example, the nurse assigned to that room may be required to handle the call when available. This may result in a delayed response to the call. In addition, no information is collected at the end of the interaction that can be analyzed to determine how well a hospital is performing (e.g., responsiveness).

It would therefore be beneficial to develop a system that addresses issues regarding integration of disparate systems to improve how information is communicated in a hospital setting.

### SUMMARY

According to one aspect, a method of initiating and/or routing communications within a system comprised of a plurality of disparate sub-systems is provided. The method includes, the method includes collecting information from one or more sub-systems regarding participants in the integrated system and receiving a communication initiated by one of the sub-systems. The method further includes identifying an event within the received communication and initiating a workflow plan based on the identified event, wherein the workflow plan utilizes collected information to generate workflow-driven communications to one or more participants. The workflow-driven communication is delivered to the one or more participants.

According to another aspect, a workflow-driven communications system for a healthcare system is provided that includes at least one input interface, at least one output interface, a database, an event hub, and a workflow module. The input interface is configured to communicate with one or more external sub-systems to collect information regarding participants within the healthcare system and to receive communications initiated by one or more participants within the healthcare system. The at least one output interface is configured to provide workflow-driven messages to the one or more participants; a database configured to store information collected from the one or more external sub-systems. The event hub is connected to the at least one input interface, wherein the event hub receives information collected from the one or more external sub-systems and stores the collected information to the database and wherein the event hub receives communications initiated by one or more participants within the healthcare system and detects events within the received communications. The workflow module is configured to receive events detected by the event hub, wherein the workflow module selects a workflow to initiate based on the event detected and utilizes the information collected from the one or more external sub-systems to generate workflow-driven messages. The workflow-drive messages are then communicated to the one or more participants via the at least one output interface.

### BRIEF DESCRIPTION OF THE DRAWINGS

FIG. 1 is a block diagram illustrating a workflow-driven communications platform according to an exemplary embodiment.

FIG. 2 is a flowchart illustrating workflow-driven communications within an integrated system according to some embodiments.

### DETAILED DESCRIPTION

According to some embodiments, the present disclosure provides a cloud-based platform for providing work-flow driven communications within an integrated communications system. In particular, the platform collects data from a plurality of disparate sub-systems included as part of the integrated system. In a medical setting, sub-systems may include hospital enterprise systems (e.g., electronic health records (EHR), physician schedule databases, staff directories, etc.). Information is collected from the disparate sub-systems and organized into a cloud-based directory. Communications initiated by one of the sub-systems is provided to the cloud-based platform, which detects events within the received communication. In response to a detected event, a workflow plan within the cloud-based platform is initiated to handle the event. The workflow plan accesses the aggregated directory to make decisions regarding how the event is handled, and specifically how information related to the response is communicated to participants within the integrated communications system. For example, decisions may include which staff members are part of the care team, which members should be contacted to respond to an event, and which members are currently working or are closest to the alarm. This information is stored in the aggregated directory including work schedule, current status, location, etc and is used to make such decisions. The workflow plan communicates data and/or messages as required by the workflow plan to selected individuals (e.g., nurses, physicians, patients) and monitors responses to determine whether alternative communications are required. In this way, the cloud-based platform provides workflow-driven communications within an integrated system.

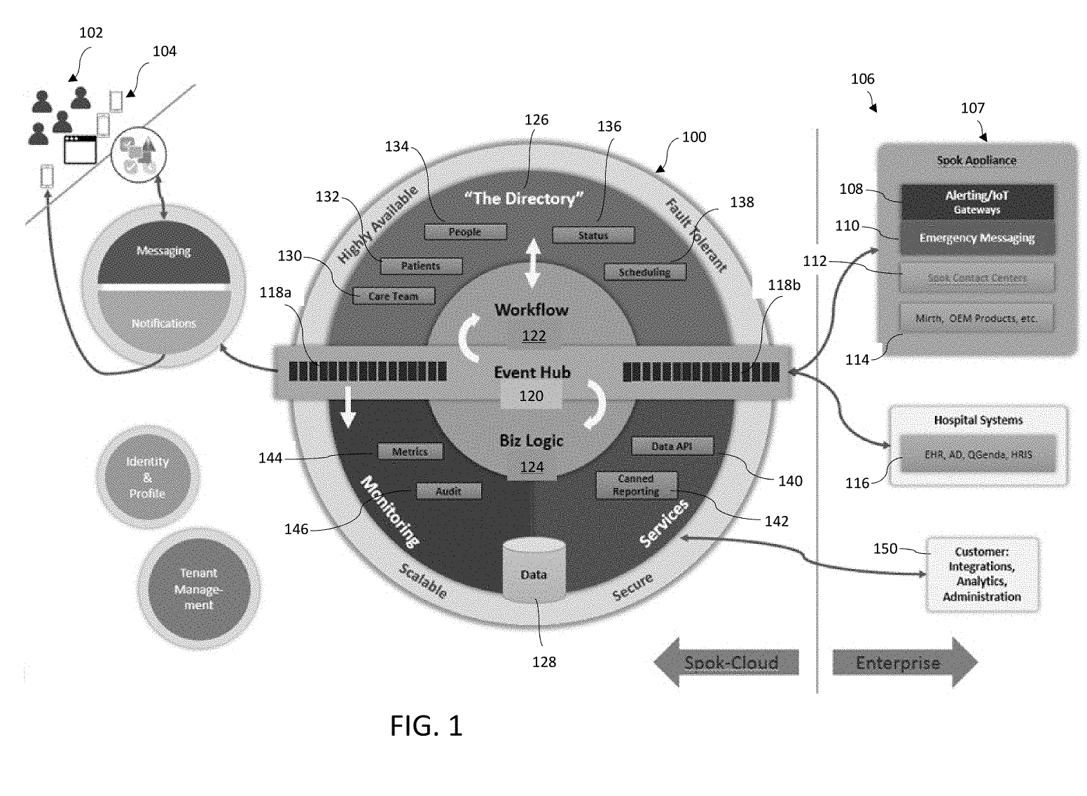

FIG. 1 is a block diagram illustrating a workflow-driven communications platform 100 according to an exemplary embodiment. Workflow-driven communications platform 100 is a cloud-based server configured to integrate communications between a plurality of disparate sub-systems and participants. Example are described with respect to a healthcare/hospital setting, in which the workflow-a plurality of disparate sub-systems are commonly employed, but is applicable to any system in which information/communications received from a plurality of disparate sources needs to be communicated to one or more participants of the system.

In the example shown in FIG. 1, participants include doctors, nurses, administrators, and patients (referred to generically as participants 102). Communications are delivered to participants 102 via one or more devices 104, including mobile devices like tablets and smartphones, smartwatches, laptops, computers, and others. In addition, communication may be received from the one or more participants 102 via the one or more devices 104. In addition to participants 102, a plurality of disparate sub-systems are provided. In the embodiment shown in FIG. 1, for example, sub-systems include enterprise systems 106 employed in a hospital setting. For example, in some embodiments, enterprise systems may include 3^{rd} party systems 107 (e.g., Spok appliances, but may include other 3^{rd} party application as well), including alerting/monitoring/internet of things (IOT) gateways (e.g., heart rate monitors attached to a patient) 108, emergency messaging systems (e.g., 911 systems) 110, contact centers (e.g., hospital operator) 112, and other 3^{rd}-party applications 114 (e.g., Mirth, OEM products, etc.). In addition, workflow-driven communications platform 100 is configured to interact and communicate with hospital enterprise systems 116 (e.g., electronic health records, physician scheduling, staff directories, etc.). In this way, the workflow-driven communications platform 100 is capable of communicating with a plurality of external systems, referred to herein generically as third-party communications.

In some embodiments, the workflow-driven communications platform 100 collects information from one or more of the disparate sub-systems, including information collected associated with one or more of the participants 102 of the system. For example, in a hospital application., one of the sub-systems (e.g. hospital system 116) may include a work schedule at least for some of the participants. This information may be communicated and stored by the workflow-driven communications platform 100. In addition to collecting information from one or more of the sub-systems, the workflow-driven communications platform 100 receives communications from the one or more sub-systems. For example, participants 102 may generate messages and/or notifications, 3^{rd} party systems such as alert/monitoring devices (e.g., heart-rate monitor) 108 may generate alerts regarding the status of a patient, emergency messaging systems (e.g., 911) 110 may generate alerts/messages regarding patient status, and hospital systems 116 such as electronic health records may be updated by a participant, wherein the updating or addition of a health record constitutes a message and/or notification. The workflow-driven communications platform 100 is connected to receive communications from the one or more sub-systems. In response, workflow-driven communications platform 100 detects events in the received communications and initiates workflow plans, wherein the workflow plans rely on information communicated and stored by the platform regarding the participants to generate workflow-driven communications to one or more of the participants.

In the embodiment shown in FIG. 1, workflow-driven communications platform 100 includes at least one processor and memory (not shown), for storing instructions and data. The at least one processor executes instructions stored by the memory to implement a plurality of logical modules/components, including one or more of interfaces 118a, 118b, an event hub module 120, a workflow module 122, a business logic module 124, and a directory 126 for storing information. The one or more interfaces 118a, 118b provide secure, bi-directional communication with one or more of the participants and communication platforms. In some embodiments, interfaces 118a, 118b are configured to convert messages into a format corresponding with the communication protocol being utilized, or corresponding with the protocol format utilized by the participant and/or communication platform (e.g., messaging system associated with devices 104). Similarly, interfaces 118a, 118b convert incoming messages received from participants and/or communication platforms into a message format that can be utilized by the workflow-driven communications platform 100.

The workflow-driven communications platform 100 supports secure, mobile messaging/communications between the workflow-driven communications platform 100 and the various types of devices 104 utilized by participants 102. Communication may include messages and/or notifications/alerts. In some embodiments, messages may include text voice and/or data. Communication is bi-directional, allowing participants to send messages and/or notification/alerts to the workflow-driven communications platform 100. As discussed in more detail below, messages and/or notification/alerts may result in the detection of an event by the workflow-driven communications platform 100 that initiates one or more actions, including generation of messages and/or notifications provided to one or more of the plurality of devices. In addition to messages, communications platform may also receive messages in the form of updated data that requires a response. For example, a physician may update a patient's (EHR with lab results. In some embodiments, the physician reviewing the lab results may generate a message with the attached lab results and provide it to the communications platform to initiate an event. In other embodiments, however, the updated patient's EHR (or at least the lab result portion) is communicated to the communications platform 100, wherein the lab result may result in identification of an event that initiates communication with one or more participants.

In addition to providing secure, mobile messaging/communications between workflow-driven communications platform 100 and the various types of devices 104, workflow-driven communications platform also provides for secure bi-directional communication with enterprise systems (i.e., information technology systems) utilized by each hospital. Communication may include retrieval of information from the one or more enterprise systems for the purpose of populating the directory 126, or detecting events based on the received communication that results in the initiation of workflow plans. As discussed in more detail below, information collected by the workflow-driven communications platform 100 with respect to one or more participants is utilized by the workflow plan to determine how to distribute messages within the integrates system.

Event hub module 120 is connected via interfaces 118a, 118b to receive inputs from the plurality of disparate sources described above, including from one or more devices 104, and enterprise systems such as hospital enterprise systems 116 and/or 3^{rd} party systems 106. Based on received inputs, event hub module 120 detects events. For example, an event may be detected in response to a physician updating a patient's electronic health record (EHR) based on received test results. The update to the patient's EHR is communicated from hospital enterprise system 116 to event hub module 120. In other embodiments, a patient alerting/IoT gateway 108 (e.g. a patient monitoring device) initiates a warning predicting a possible heart attack. The message is communicated to event hub module 120, which detects/identifies the event. Similarly, events may be generated in response to message communicated from one of the plurality of devices 104, such as message from a physician indicating the results of a test. In this way, event hub module 120 identifies events from a plurality of different sources in a plurality of different formats, including messages, changes to electronic health records, alerts, voice calls, etc.

Workflow module 122 receives the detected events and generates/selects in response a workflow plan to initiate to handle the event. In some embodiments, the workflow plan analyzes data stored by the directory to formulate the workflow-driven communications. In some embodiments, workflow module 122 analyzes data stored in the directory with respect to one or more participants to formulate and generate workflow-driven communications. That is, workflow module 122 may interact with one or more databases associated with directory 118 to determine how to respond to a detected event, wherein the response includes generating additional events (e.g., communication events). For example, an update to an electronic health record (EHR) detected by event hub module 120 indicating a critical condition. In response to this event, the workflow module 122 accesses the directory 118 (specifically, information associated with the patient's primary care physician) and initiates a secure message to the patient's primary care physician alerting the physician to the critical condition. The patient's physician may respond to the message indicating a course of action, initiating another event to be handled by the event hub module 120 and workflow module 122. Conversely, if no response is received from the physician within a set amount of time, the workflow module accesses the directory 126 to identify a physician on-call (e.g., accessing the scheduling database stored in the directory 126) and initiates a secure message to the second physician alerting the physician of the critical condition. In this way, the workflow module determines (i.e., drives) the communications within the integrated system.

In other example, an alert is communicated by alerting/IoT gateway 108 predicting a potential heart attack for a particular patient. Event hub module 120 detects the event and workflow module 122 interacts with the directory 126 to initiate a workflow plan. For example, this may include accessing the schedules of available participants (e.g., physicians and/or nurses), as well as the status of the available participants (e.g., available, busy non-critical, busy critical, etc.). Based on the collected information, the workflow module 122 selects one or more participants to review a message/communication with respect to the detected event. Event hub module 120 generates secure messages to the selected participants, wherein the secured message includes information regarding the particular patient (e.g., name, location, condition detected, etc.) along with a message regarding the action required. In some embodiments, the message may be sent to a first plurality of participants, and based on responses received regarding availability (creating additional events), workflow module 122 may select additional participants (e.g., alternates) and event hub module 120 may then generate secure messages to the additional participants until a team is assembled to handle the event. In this way, communication within the integrated system is driven by the workflow module 122. Each of the events—including the detected events and generated events—are stored in database 128 and can be subsequently analyzed to determine metrics, audit performance, etc.

In some embodiments, the directory 126 is a database or plurality of databases accessed by the workflow module 122 to make decisions regarding how to respond to a particular event. For example, the one or more databases may include a care team database 130, patient database 132, staff database 134, status database 136 and scheduling database 138. These examples are merely representative, and other embodiments may include fewer or additional databases, and/or may combine some of the databases into a single database. These databases may be populated based on information provided by third-party databases, for example information may be retrieved from hospital enterprise systems 116 (e.g., staff directories) and/or other sources. That is, while the information is stored in the cloud-based workflow-driven platform, it may be based on information stored locally by individual hospital IT systems.

In one embodiment, care team database 130 stores information/records regarding a currently assigned team of doctors, nurses, etc. Assignment may be associated with individual patients, particular departments, physical locations within a hospital, or other features. Participants (e.g., doctors, nurses) may be associated with a plurality of care teams, and the database may be searchable by any of the plurality of records. For example, an event detected with respect to a particular patient may result in workflow module 122 generating a message to all participants of the patient's care team informing them of the event. In other embodiments, an event detected with respect to a patient located in a particular room (e.g., room 204) may result in workflow module 122 generating a message to all participants of a care team associated with the patient's room. Similarly, the patient database 132 stores information/records regarding patients, including patient location (e.g., GPS location, assigned room, etc.), patient status, contact info for the patient and/or family, etc. For example, in one embodiment, in addition to assembling a team of participants to handle a detected event, the workflow module 122 may also interact with patient database 132 to identify contact information for a spouse or other family member to notify them of the event. In some embodiments, the staff database includes contact information for all staff members. For example, this may include information regarding the role or job of each member, as well as contact information. In particular, contact information may include not only email address, phone number of the staff member, but may also include information regarding preferred method of contact, as well as particulars regarding the particular platform utilized by the participant for communication. For example, one staff member may prefer getting instant text messages sent to a mobile device, while another staff member may prefer getting instant messages sent to a particular account. In some embodiments, status database 136 maintains information regarding the availability/status of each participant. For example, if a particular member is in surgery, the status may indicate this and as such may be utilized to determine that the surgeon is not available. Similarly, scheduling database 138 maintains a schedule (e.g., work schedule) of all members. For example, in response to a detected event, workflow module 122 may access scheduling database 138 to determine which staff members are available. In some embodiments, the databases maintained by the directory 126 are accessible by authorized users of the systems, allowing participants to update information directly to the one or more databases. For example, in some embodiments authorized users are allowed to update schedule information and/or contact information associated with themselves and/or other participants of the system. In addition, participants may access the database to retrieve information directly. For example, an authorized participant may access the directory 126 to retrieve contact information to send a message to another participant in the system.

In some embodiments, event hub module 120 and workflow module 122 initiate events to maintain the integrity of information stored to the directory 118. In other words, stored data is “cured” to make sure the data stored is accurate. In some embodiments, data cure operations are initiated as independent events, provided to workflow module 122 for execution. An example of the former, an event may be initiated that sends a message to all staff members using stored contact information and requests a response via a preferred method. As a result, contact information that is no longer valid is identified and deleted and other contact information is updated. In another example, messages include location data (e.g., GPS data) that can be utilized to determine status and/or availability of a staff member to respond to a critical threat. For example, the scheduling database 138 may indicate that a particular physician is working; however, due to an unexpected event is not physically at the hospital. Based on location information associated with a message sent by the physician, the directory 118 is updated to reflect the unavailability of the physician in the status database 136 and/or scheduling database 138. Similarly, if in response to a detected event a message is sent to a staff member, but no response is received in reply, then in addition to escalating the event response to another staff member, the status of the physician is modified to indicate current unavailability. Other examples of changing the current availability of the staff member include the mobile operating system companies (e.g, Apple, Google) notifying the platform that a particular device is no longer valid, calling a phone number and receiving an invalid phone number recording, using real-time location based services to inform the platform that the physician is not within the area, and if a staff member logs out of a web interface then the platform will know to not send the notification. Users will receive notifications when a patient has been admitted, transferred, discharged, a procedure has been ordered for a patient, the results for an order have been completed, or when there is an abnormal vital sign for a patient who is connected to a monitoring system. The hospital is able to review reports to determine average times to respond to key events, compare users response times to hospital averages, ensure alarms are being addressed within healthcare standards (e.g., JCAHO, MAGNET), and identify patterns in staff member's behavior when receiving notifications.

Business logic module 124 stores information associated with detected events and initiated workflow plans. In one embodiment, information is stored to database 128. Data application programming interface (API) 140 can be utilized to access and retrieve data from database 128. In addition, canned reporting module 142 provides pre-built reports built from data stored in database 128. For example, canned reports may include how many patient rooms are empty at any given time, or a current usage rate of staff to determine if additional staff needs to be called in. Metrics module 144 similarly accesses events and initiated workflow plans stored to database 128 to analyze performance. For example, the average response time of an assembled team in response to a heart attack or similar critical event. In this way, stored data associated with detected events and initiated workflow plans as well as reports generated based on this stored data, may be communicated to the customer or third party for review (e.g., communicated to an administrator 150).

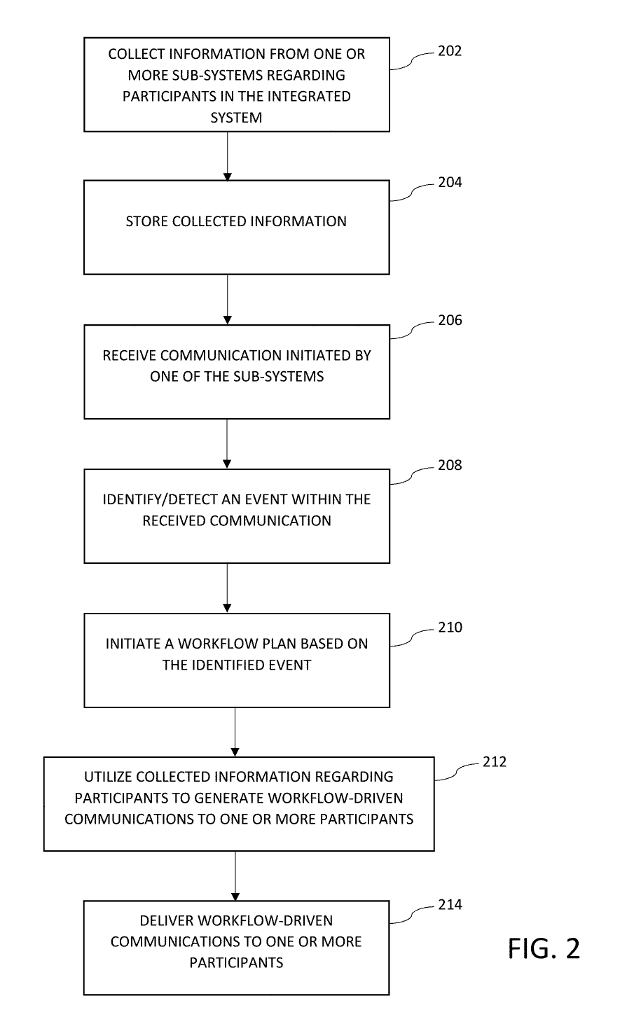

FIG. 2 is a flowchart illustrating workflow-driven communications within an integrated system according to some embodiments.

At step 202, information is collected from one or more sub-systems regarding participants in the integrated system. As discussed above, information may be collected from one or more of a plurality of databases. In one embodiment, information collected with respect to participants includes one or more of preferences as to how messages are delivered to the participant (e.g., text message, email, instant message alert), the work schedule of the participant, status of the participant (e.g., busy, available), specialty associated with the participant, department, patients associated with the participant, etc. In non-medical applications, different criteria related to the participants may be required, including clearance level, work schedule, etc.

At step 204, the collected information is stored by the workflow-driven communications platform. In some embodiments, the workflow-driven communications platform is implemented as a cloud-based server, wherein the collected information is stored as part of the cloud-based implementation. In other embodiments, the collected information is stored separately, and accessed by the workflow-driven communication platform on demand.

At step 206, communications initiated by one or more of the sub-systems are received and/or monitored by the workflow-driven communications platform. As discussed with respect to FIG. 1, interfaces 118a, 118b are configured to receive messages/communications initiated by the one or more sub-systems. As discussed above with respect to FIG. 1, initiated communications may be a result of updates made to a patient's electronic health record (EHR), or may be in response to an alert received from a device monitoring patient health, as well as a number of other sources.

At step 208, an event is identified/detected within the received communication. As discussed with respect to FIG. 1, event hub module 120 is detects/identifies events based on received communications. For example, an alert detected indicating a patient is going into cardiac arrest may be interpreted as a critical event, or crash team event. In another example, the event identified may be non-critical lab test results.

At step 210, a workflow plan is initiated by workflow module 122 based on the event identified/detected by event hub module 120. Using the examples provided above, in response to a detected critical event (cardiac arrest of a patient), the workflow plan may be to send urgent alerts to one or more participants to drop everything and assist the patient. Conversely, if the event is identified as a non-critical lab test result, the workflow plan may be to communicate the results to the patient's primary care physician for subsequent disclosure to the patient, or may be to send a message directly to the patient with either details regarding the lab test result, or instructions to contact the patient's physician to discuss the results, with no timetable or requirement that a response be received within a certain amount of time due to the non-critical nature of the results. The specific workflow plan initiated by workflow module 122 based on the identified/detected event may be modular, customizable and programmed in advance of the identification/detection. For example, the workflow plan may be developed and/or programmed by a third-party, operating outside of the integrated communications system of FIG. 2, the third-party having access to a larger history of relevant data and/or access to data specifying critical times for response or treatment regiments (e.g., based on real-world data sets and/or predictive models) that is not directly available in or with the integrated communications system of FIG. 2. Such workflow modules may be provided (e.g., sold or given) to an operator of the integrated communication system of FIG. 2 for use in the system. The use of customizable and modular workflows allows for more effective outcomes (e.g., treatment of patients) than would be possible otherwise.

At step 210, collected information regarding the one or more participants is utilized to generated workflow-driven communications to the one or more participants. Continuing the above examples, a critical event requiring urgent alerts be sent to one or more participants to aid the patient may utilize collected information to determine which participants are scheduled to work, and/or may further determine which participants are located in close proximity to the patient, and/or may further determine which participants have a status set to a level less-critical than the current event, and/or the preferred method of contacting the participant (e.g., phone, tablet, messaging system, text message, etc.). Depending on the collected data available, additional criteria may be utilized to determine those participants that should receive a given communication.

At step 212, the workflow-driven communications are delivered to the one or more participants. Response or lack of responses to the delivered communications may in themselves be identified as events that require communication to one or more participants. In this way, the present disclosure provides a system and method for providing work-flow driven communications within an integrated communications system.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of initiating and/or routing communications within a system comprised of a plurality of disparate sub-systems, the method comprising:
collecting information from one or more sub-systems regarding participants in the system;
receiving a communication initiated by one of the sub-systems;
identifying an event within the received communication;
initiating a workflow plan based on the identified event, wherein the workflow plan utilizes collected information to generate workflow-driven communications to one or more participants; and
delivering the workflow-driven communications to the one or more participants.

2. The method of claim 1, wherein collected information includes work schedules for the one or more participants.

3. The method of claim 2, wherein initiating the workflow plan based on the identified event includes utilizing the collected work schedules to determine that each of the one or more participants should receive the workflow-driven communication.

4. The method of claim 3, wherein collecting information from one or more sub-systems further includes collecting information regarding location of the one or more participants.

5. The method of claim 4, wherein location of the one or more participants is based on global positioning system (GPS) data collected from a respective device associated with each of the one or more participants.

6. The method of claim 4, wherein initiating the workflow plan further includes utilizing the location of the one or more participants to determine that each of the one or more participants should receive the workflow-driven communication.

7. The method of claim 1, wherein collected information includes a status level associated with each of the one or more participants.

8. The method of claim 7, wherein initiating the workflow plan further includes utilizing the status level associated with the one or more participants to determine that each of the one or more participants should receive the workflow-driven communication.

9. The method of claim 1, wherein collecting information from one or more sub-systems regarding participants in the system includes collecting a preferred method of communication with respect to each of the plurality of participants.

10. The method of claim 9, wherein generating workflow-driven communications includes utilizing the preferred method of communication associated with each of the plurality of participants.

11. The method of claim 1, wherein receiving a communication initiated by one of the sub-systems includes one or more of messages received from one or more participants, updates to a patient's electronic health record (EHR), and alerts generated by a patient monitoring device.

12. The method of claim 1, further including monitoring responses to workflow-generated messages and initiating one or more additional messages if no response is received within a determined length of time.

13. The method of claim 1, wherein information collected from one or more sub-systems is compared to information collected from another sub-system to verify status and/or availability of one or more participants.

14. The method of claim 13, wherein global positioning system (GPS) information received from devices associated with one or more participants is compared with work schedule information received from a sub-system to verify the availability of the one or more participants.

15. A workflow-driven communications system for a healthcare system, the communication system comprising:
at least one input interface configured to communicate with one or more external sub-systems to collect information regarding participants within the healthcare system and to receive communications initiated by one or more participants within the healthcare system;
at least one output interface configured to provide workflow-driven messages to the one or more participants;
a database configured to store information collected from the one or more external sub-systems;
an event hub connected to the at least one input interface, wherein the event hub receives information collected from the one or more external sub-systems and stores the collected information to the database, wherein the event hub receives communications initiated by one or more participants within the healthcare system and detects events within the received communications; and
a workflow module configured to receive events detected by the event hub, wherein the workflow module selects a workflow to initiate based on the event detected and utilizes the information collected from the one or more external sub-systems to generate workflow-driven messages, wherein workflow-drive messages are communicated to the one or more participants via the at least one output interface.

16. The communications system of claim 15, wherein information collected from the one or more sub-systems includes work schedules for the one or more participants.

17. The communications system of claim 16, wherein the workflow module utilizes the stored work schedules to determine that each of the one or more participants should receive the workflow-driven communication.

18. The communications system of claim 16, wherein information collected from the one or more sub-systems further includes information regarding location of the one or more participants.

19. The communications system of claim 18, wherein location of the one or more participants is based on global positioning system (GPS) data collected from a respective device associated with each of the one or more participants.

20. The communications system of claim 19, wherein the workflow module utilizes the location of the one or more participants to determine that each of the one or more participants should receive the workflow-driven communication.
